(19) United States
(12) Patent Application Publication
(10) Pub. No.: US 2012/0022666 A1
(43) Pub. Date: Jan. 26, 2012

(54) Actuator for Prosthetic Finger and Method

(76) Inventors: Adam W. Brooks, Woolford, MD (US); Scott H. Bouwer, Wilmington, DE (US)

(21) Appl. No.: 13/143,367

(22) PCT Filed: Jan. 6, 2010
(86) PCT No.: PCT/US10/20174

§ 371 Date: Jul. 6, 2011

Related U.S. Application Data
(60) Provisional application No. 61/142,923, filed on Jan. 7, 2009.

(51) Int. Cl. A61F 2/48

(52) U.S. Cl. 623/24

(57) ABSTRACT

A prosthetic glove worn over damaged fingers or over a prosthetic hand having flexible fingers. The glove articulates the fingers with multiple shape memory alloy wires and includes a cooling system for each shape memory alloy wire.

### FIELD OF THE INVENTION

The invention relates to an actuator for a finger to control movement of the finger in response to an input signal and to related methods. The finger is moved by contraction of a shape memory alloy wire. The actuator can move a biological finger not moveable by finger muscles or can move a prosthetic finger.

### BACKGROUND OF THE INVENTION

Loss of finger function severely affects an individual's ability to perform manual tasks.

Prior prosthetic devices have restored limited hand function to individuals unable to control finger movement. These devices use robotic technologies and actuate fingers through use of motors or other mechanisms.

Prosthetic devices have used shape memory alloy wires made of Nitinol or like materials as actuating members. The Nitinol wire forms part of an electrical circuit that selectively flows current through the Nitinol wire to heat the wire to a transition temperature that contracts the wire. The wire contraction actuates a member joined to a prosthetic or otherwise uncontrollable finger to move the finger.

The use of shape memory alloy wires to actuate prosthetic devices is difficult. A long Nitinol wire is required to generate a sufficient contraction stroke for finger actuation. Heated Nitinol wire only shrinks about 4% of its unheated length. The long wire length necessitates prior prosthetic devices to include long support portions to accommodate the long shape memory alloy wires.

Another problem with prior prosthetic devices is reliance on radiation cooling of the hot, contracted wire below the transition temperature to return the wire to its original length prior to another contraction. Radiation cooling slows lengthening and delays finger contraction.

Accordingly, there is a need for a compact actuator for a finger that uses shape memory alloy wire which rapidly cools and re-lengthens the contracted wire.

### SUMMARY OF THE INVENTION

The invention is an actuator worn by an individual with a prosthetic finger or an otherwise uncontrollable finger to control movement of the finger. Tendon or tension wires are connected to the finger. An increase in the tension in one wire opens the finger. Tension in another wire closes the finger.

The end of each tendon wire away from the finger is connected to a shape memory alloy (Nitinol) actuating wire. Each actuating wire extends from the tendon wire through a hollow tube to an anchor to form a wire and tube assembly that is wound in a compact coil. The ends of the actuating wire are connected to an electrical circuit which, in response to a signal, flows current through the wire to heat the wire to a transition temperature in order to shorten the wire and tension the tendon wire for resultant movement of the finger.

After heating and shortening of the wire, the wire is rapidly re-lengthened by gas cooling. This is achieved by flowing a gas, such as carbon dioxide, into the coiled tube and along the wire to quickly cool the wire below the transition temperature, re-lengthen the wire and prepare the wire for subsequent shortening in response to another input signal and current flow through the wire.

Actuation of a wire to flex a finger in one direction pulls out a previously retracted and cooled wire used to move the finger in the opposite direction to lengthen the wire for reheating and contraction in response to another signal.

The actuator may include sensors on the fingertip to actuate circuits to heat and retract Nitinol wires actuating the finger.

Individual wire and tube actuators may be wound around a cylindrical support that surrounds the wrist supporting the finger or may be stacked on the back of a hand supporting the finger. The actuators may be wound in flat spirals.

A number of wire and tube actuators may be provided for the finger so that the finger is moved independently about finger joints in response to signals from finger sensors. A sensor on the end of the finger actuates an actuator or actuators to flex the finger inwardly in a gripping motion. Actuators may also move a finger laterally to either side in response to signals. In the absence of a flex signal, a control system automatically releases or straightens the finger.

### DESCRIPTION OF THE DRAWINGS

FIG. 1 is a side view of the invention on a hand;

FIG. 2 is a top view of FIG. 1;

FIG. 3 is a detail view of invention support plates and tension wires;

FIG. 4 is a representational view of the actuator with the tube and wire unwound;

FIGS. 5, 6, 7, and 8 are detail views of a distal support member;

FIGS. 9, 10 and 11 are detail views of a medial support member;

FIGS. 12, 13, and 14 detail views of a proximal support member;

FIGS. 15 and 16 are perspective and top views of a casing assembly;

FIG. 17 is a sectional view taken along line 17-17 of FIG. 16;

FIG. 18 is a perspective view of a wrist-surrounding casing assembly;

FIG. 19 is a view showing the wrist-surrounding casing assembly taken along line 19-19 of FIG. 18;

FIG. 20 is a sectional view of the wrist-surrounding casing assembly taken along line 20-20 of FIG. 19;

FIG. 21 is a sectional view of the wrist-surrounding casing assembly along line 21-21 of FIG. 20; and

FIG. 22 is a sectional view of the wrist-surrounding casing assembly taken along line 22-22 of FIG. 21.

### DESCRIPTION OF THE INVENTION

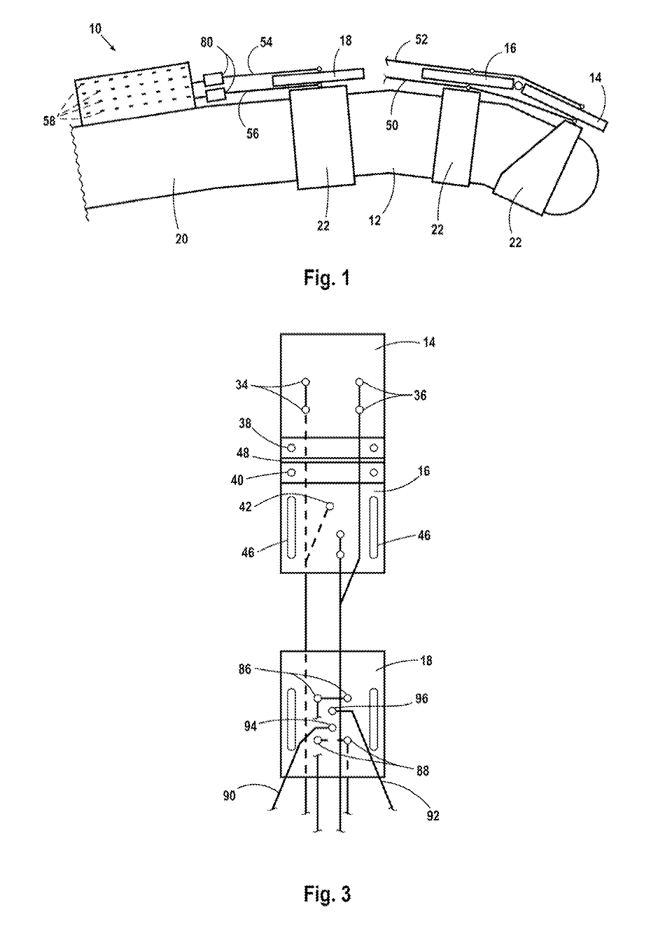

As illustrated in FIG. 1, first embodiment prosthetic glove 10 includes a finger sheath 12 with flat distal support member or plate 14, flat medial support member or plate 16 and flat proximal support member or plate 18 secured to the top of the sheath. Palm portion 20 of glove 10 covers the palm supporting the finger in sheath 12.

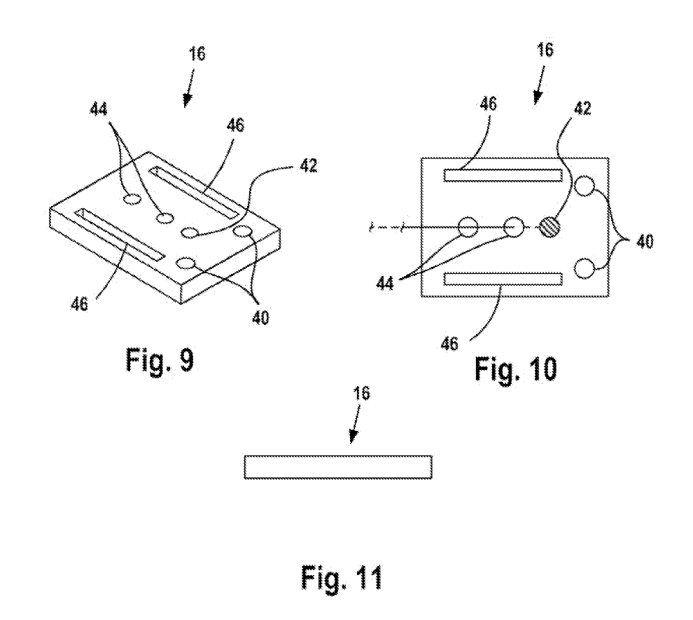

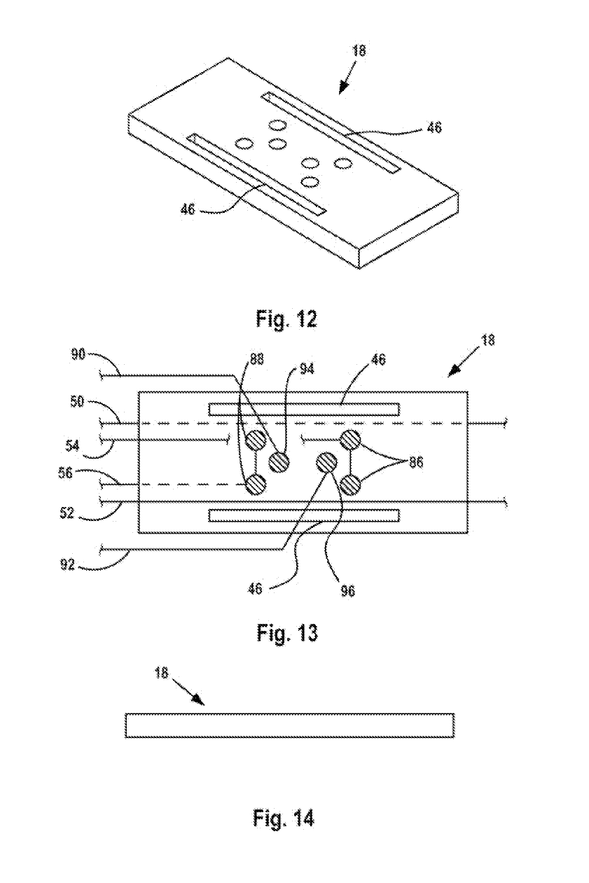

Support members 14, 16 and 18 may be plates as shown in profile in FIG. 1 and in alternate views in FIGS. 9, 10, 11 12, 13 and 14. The plates are bound to the top of finger sheath 12 by bands 22 attached to each plate and surrounding finger sheath 12.

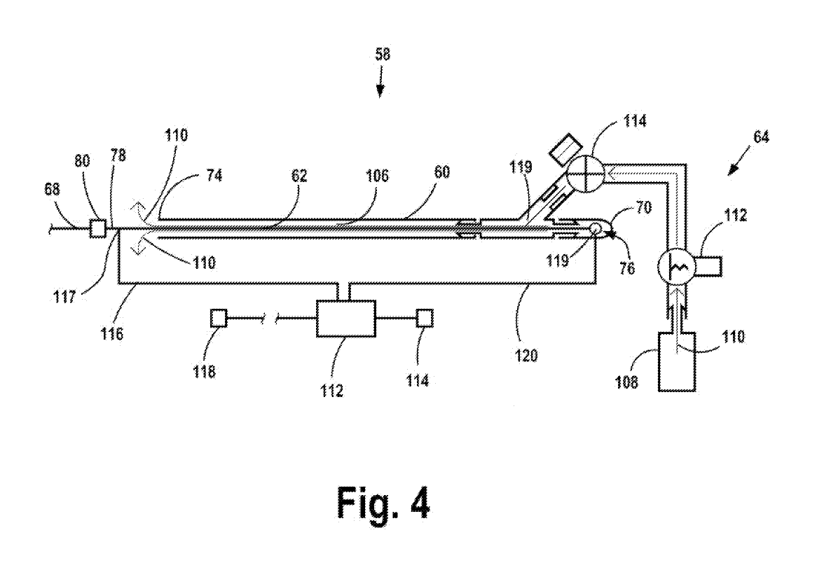

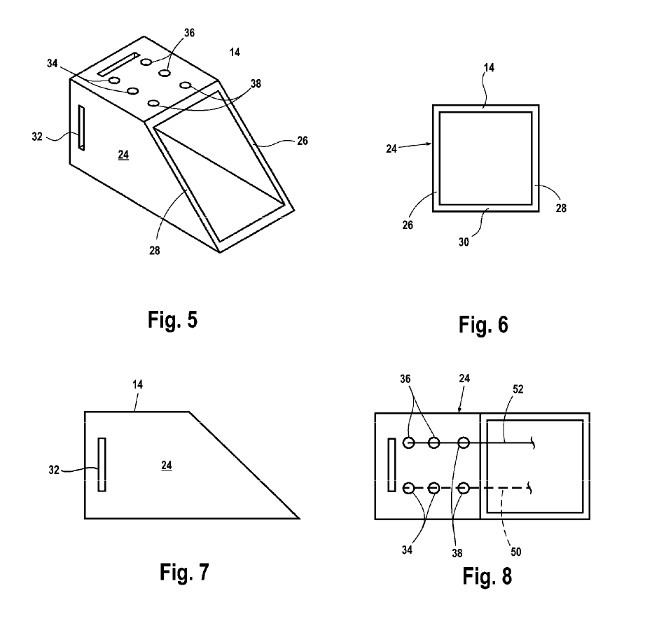

In an alternative embodiment, each support member 14, 16 or 18 may form one wall of an open ended square tube 24 as illustrated in FIGS. 5-8. Tube 24 includes flat sidewalls 26 and 28 joining support 14 and flat bottom wall 30. In member 14, sensor slots 32 are provided in the lead end of the member 14 of walls 26 and 28. The slot in wall 28 is not illustrated. Pressure sensors may be mounted in slots 32.

Two pairs of tendon wire-mounting holes 34 and 36 are located on opposite sides of support 14. Hinge mounting holes 38 extend through the proximal end of the member.

Medial support member 16 includes two hinge mounting holes 40 on the distal end thereof, a tendon wire mounting hole 42 and a pair of tendon wire mounting holes 44. Holes 42 and 44 are on the centerline of the member. Band attachment slots 46 extend along opposite sides of the support member.

Hinge 48 is connected to support members 14 and 16 at holes 38 and 40 to permit relative rotation of the members during flexing of the finger and sheath 12.

The three support members 14, 16 and 18 are mounted on finger sheath 12 as illustrated in FIG. 1 so that member 14 overlies a finger's distal phalanges bone, member 16 overlies a finger's middle phalanges bone and member 18 overlies a finger's proximal phalanges bone. A band 22 may be secured to member 14, 16 or 18 by band attachment slots 46.

In embodiments in which support members 14, 16 or 18 form parts of square tubes 24, as shown with a support member 14 in FIGS. 5-8, sheath 12 extends into the interior of the tubes so that the support member overlies the top of the finger.

Finger sheath 12 is closed and opened to grip and release objects by selectively tensioning and relaxing tendon wires 50, 52, 54 and 56 connected to members 14, 16 and 18 as illustrated in FIGS. 1, 2 and 3. Each tendon wire is connected to a wire actuator 58 having a coiled dielectric tube 60 and a shape memory wire 62 in the tube. An electrical circuit flows current through the shape memory alloy wire to heat and contract the wire and a gas supply assembly 64 flows cooling gas through the tube to cool and elongate the heated wire 62. The end of the shape memory alloy wire away from the tendon wire is fixed in position. The other end of the shape memory alloy wire is connected to the tendon wire to tension and relax the tendon wire.

Individual wire actuators 58, each with a coil of Nitinol shape memory alloy wire in a coiled tube, are located in housing 66, secured to the back of the glove 10 above the palm of the glove. See FIG. 1.

The housing 66 surrounds each coiled wire actuator 58 for each tendon wire 68 attached to a member 14, 16 or 18. Each wire actuator 58 has a coiled dielectric tube 60 with a fixed closed end 70, a coiled length 72 and a fixed open end 74. A length of memory alloy wire 62 extends the length of the interior of the tube from a fixed wire end 76 at tube fixed closed end 70, along the tube, past open end 74, which may be fixed, to wire end 76 outside the tube where the end of the memory shape alloy wire is joined to the proximal end 78 of a tendon or tension wire 68 wire outside of the tube at a turnbuckle or length-adjusting device 80. FIG. 4 shows an uncoiled wire actuator 58.

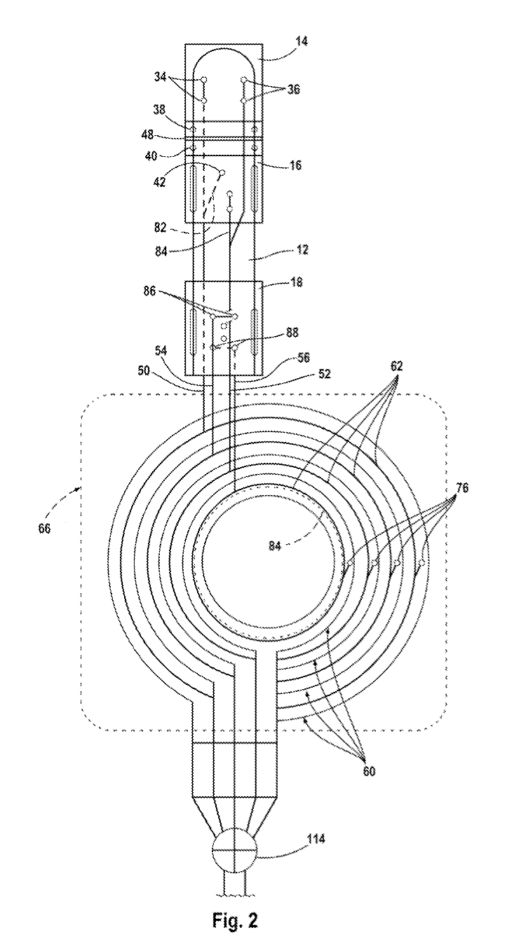

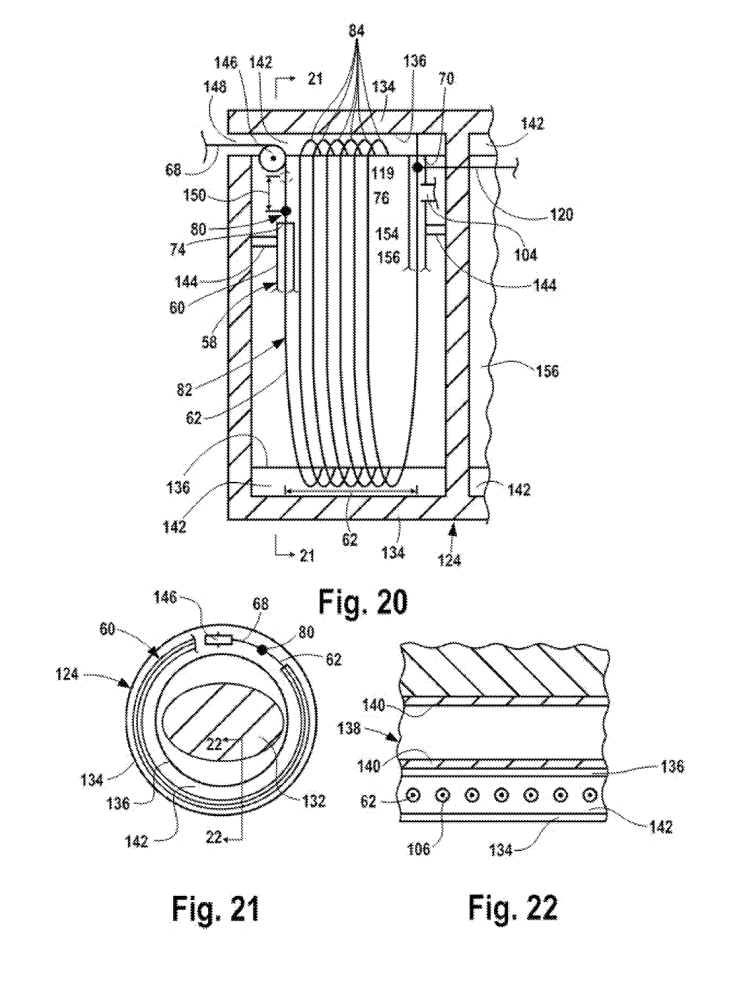

FIG. 2 illustrates the three support members 14, 16 and 18, the tendon wires connected to the members and a housing 66 containing multiple wire actuators 58. Each wire actuator 58 is wound into a coil made up of one or more coil loops. The actuator may be wound into a flat coil having co-planar loops as illustrated in FIGS. 1 and 2. The actuator may be wound into a cylindrical coil having circular loops arranged adjacent to each other as illustrated in FIG. 20.

Tendon wire 50 contracts members 14 and 16 to close the finger to grip an object. Tendon wire 52 extends from turnbuckle or length-adjusting device 80 adjacent housing 66 under proximal member 18 and under members 16 and 14 to an end secured to mounting holes 34 in member 14. A branch 82 of tendon wire 50 is joined to tendon wire 50, extends under member 16 and is connected to member 16 at hole 42.

Tendon wire 52 opens members 14 and 16 and extends from turnbuckle or length-adjusting device 80 adjacent housing 66 over member 18, and over member 16 and 14 to an end attached to holes 36 in member 14. A tendon wire branch 84 joins tendon wire between members 16 and 18, extends over member 16 and is connected to holes 44 in member 16.

Tendon wire 54 extends from turnbuckle or length-adjusting device 80 adjacent housing 66 across the top of member 18 and is connected to the top of the member at holes 86. Tendon wire 56 extends from turnbuckle or length-adjusting device 80 adjacent housing 66 under member 18 and is connected to the bottom of member 18 at holes 88. Tensioning of tendon wire 54 rotates member 18 upwardly to open the finger. Tensioning of tendon wire 56 moves the member 18 downwardly to close the finger.

The finger sheath is moved laterally by tensioning tendon wires 90 or 92. Tendon wire 90 extends from a turnbuckle or length-adjusting device 80 adjacent housing 66 over member 18 and is connected to the top of the member at hole 94. Tendon wire 92 extends from a turnbuckle or length-adjusting device 80 adjacent housing 66 over member 18 and is connected to the top of the member at hole 96. Tendon wires 90 and 92 extend from assembly 66 to member 18 at angles so that tensioning of either wire by the assembly exerts a torque on the finger sheath tending to rotate the finger sheath to one side or the other side, depending upon which wire 90 or 92 is tensioned. See FIGS. 3 and 13.

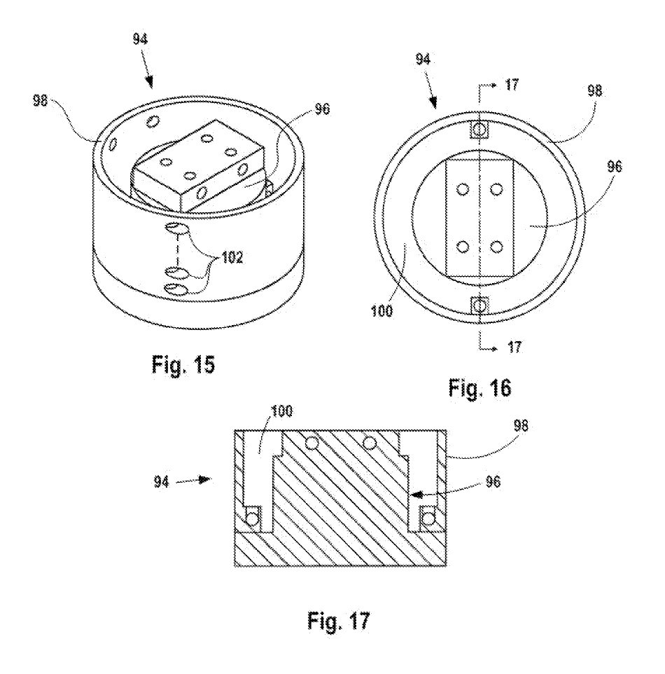

Housing 66 includes casing assembly 94 shown in FIGS. 15-17. Assembly 94 includes a central cylindrical post 96, an outer cylindrical wall 98 and a cylindrical recess 100 between the post and wall. Wire access openings 102 extend through wall 98 to recess 100. Casing assembly 94 includes a number of vertically stacked coiled wire actuators 58.

In each actuator 58, the fixed wire end 76 of Nitinol wire 62 is anchored to casing 94. The other end of the wire 62 extends outwardly from wire actuator 58 through an opening 102 and is connected to a tendon wire 68 at turnbuckle or length-adjusting device 80. See FIGS. 2 and 4.

Shape memory alloy wire 62 is surrounded by a flexible dielectric tube 60 to permit the flowing of a cooling gas through the tube and over the wire. The tube may be made of rubber, plastic or a like material. Gas inlet port 104 is located proximate fixed wire end 76 and joins tube interior 106 to gas supply assembly 64. The open tube end 74 forms a gas outlet port. Gas supply assembly 64 includes a high-pressure source of cooling gas 108 that releases a cooling gas 110 through solenoid valve 112 and pressure regulator 114. Cooling gas 110 may be carbon dioxide. Actuation of valve 112 flows cooling gas 110 along tube interior 106 and shape memory alloy wire 62 to cool and lengthen the heat contracted wire and to outlet port 74. Thermocouple 112 is mounted to tube 60 mid-way along the length of the tube. The thermocouple is connected to a controller 114 for wire 62 as described below.

The ends of each shape memory alloy wire adjacent to fixed end 76 and device 80 are connected to an electricity source 118 and micro-controller 114 at contacts 117 and 119.

Controller 114 selectively flows electricity through contacts 117 and 119 along wire shape memory wire from end 78 to end 76. The entire length of the shape memory wire 62 is heated, thus allowing generation of a maximum contractive stroke for the wire.

Contraction and release of a finger using prosthetic glove 10 will now be described.

When the sensor in slot 30 in the bottom wall of tube 20 engages an object, a signal is sent to micro-controller 114 to contract the Nitinol wires connected to finger contraction tendon wires 50 and 56. Flow of electricity through the Nitinol wires connected to tendon wires 50 and 56 contracts the wires to contract members 14, 16 and 18. Electricity is flowed through the Nitinol wires until the wires are heated sufficiently to contract.

The wires may be heated by direct current, alternating current or a current pulse-train proportional to the force exerted on the pressure sensor. When the Nitinol wires are fully contracted, the micro-controller turns off the current flow through the wires. If the wires are not fully contracted when the sensor is deactivated and the current is turned off, a subsequent signal from the pressure sensor will reactuate the micro-controller to flow a current through the wires to further contract the wires and contract the finger.

The Nitinol wires are heated during contraction. Each wire has a maximum contraction stroke. In order to prepare heated, contracted wires for a future contraction stroke it is necessary to cool the wires below the activation temperature. This must be done rapidly in order to permit repeated actuation of the finger.

When a Nitinol wire has been fully contracted and the actuation signal to the micro controller to flow electricity through the wire is deactivated, thermocouple 112 senses the temperature of Nitinol wire 62 in tube 60. If the temperature of the wire is above the transition temperature for the wire, the micro-controller sends a signal to solenoid valve 112 to flow a cooling gas such as carbon dioxide through the length of tube 60, and past wire 62 to cool the wire below the actuation temperature. Flow of the cooling gas is stopped when the sensed temperature of the wire is below the actuation temperature.

Tendon wires 52 and 54 are likewise tensioned by flowing electricity through their respective Nitinol wires to move members 14, 16 and 18 in upward, opening movement to release the grip of the finger. Opening of the finger stretches out the previously contracted Nitinol wires for tendon wires 50 and 56. Likewise, contraction of the finger stretches out the previously contracted Nitinol wires for tendon wires 52 and 54.

Wires 90 and 92 are likewise tensioned to rotate the finger sheath from side to side. Movement of the sheath to one side stretches out the Nitinol wire for moving the sheath to the opposite side.

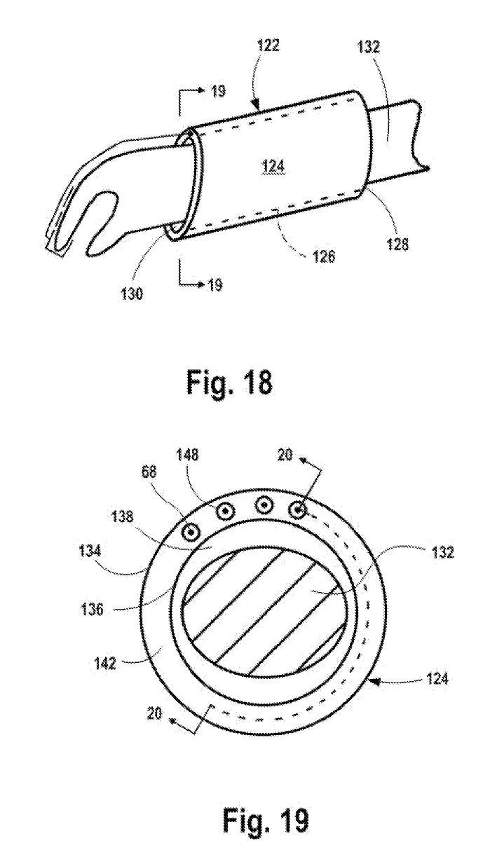

FIGS. 18 though 22 illustrate a second embodiment of the invention having a wrist-surrounding casing assembly 122. Casing assembly 122 has cylindrical body 124 defining a cylindrical passage 126 extending from an assembly rear opening 128 to an assembly front opening 130 though which a user places their wrist or arm 132. Cylindrical body 124 has an outer cylindrical wall 134 and an inner cylindrical wall 136.

Inflatable cuff 138 having cuff walls 140 is mounted on inner cylindrical wall 136 in passage 126 and is inflated to surround and comfortably fit the assembly on the user's arm 132. Outer cylindrical wall 134 and inner cylindrical wall 136 define casing assembly interior cylindrical cavities 142.

FIGS. 20 and 21 shows a coiled cylindrical tube 60 and wire 50 positioned in one interior cylindrical cavity 142. Additional actuators 50 are positioned in cavities 142 spaced along assembly 122.

Coiled actuator 58 is positioned in cylindrical cavity 142 by affixing the ends of tube 60 to mounts 144 located proximate tube closed end 70 and tube open end 74 as shown in FIG. 20. Port 104 is located proximate tube closed end 70 and connects tube interior 106 to cooling gas supply assembly 64 as previously described.

Memory alloy wire 62 extends from fixed wire end 76 along tube 60 through tube open end 74 and is joined to tendon wire 68 at turnbuckle or length-adjusting device 80. Tendon wire 68 extends about pulley 146 and out of interior cylindrical cavity 142 through assembly exit hole 148.

FIG. 20 shows memory alloy wire 62 heated above the transition temperature to a fully contracted length. As memory alloy wire 62 is cooled below its transition temperature, it will lengthen. Memory alloy wire stroke distance 150 shows the contraction distance for wire 62, illustrated as the distance moved by turnbuckle or length-adjusting device 80 from a lengthened, cooled position 154 to a contracted, heated position 152.

Walls 156 separate adjoining cylindrical cavities 142.

The second embodiment of the invention functions like the first embodiment to tension tendon wires.

1. A controller for moving a finger, the controller including an elongate tube formed from dielectric material and having opposed first and second tube ends and an interior passage extending between said tube ends; a shape memory wire having opposed wire ends, said wire extending along the interior of the tube and having a first wire end adjacent the first tube end, and a second wire end adjacent the second tube end; said first end of the tube attached to a support member and said first wire end attached to a support member; said second wire end to be attached to a tendon member for the finger; a first electrical contact on said wire adjacent the first wire end; a second electrical contact on the wire adjacent the second wire end; a circuit joined to said contacts; a gas source; a gas inlet port in the tube; a gas outlet port in the tube, said gas source connected to said inlet port; wherein actuation of the circuit flows electricity through the section of the wire between said contacts to heat the wire section above a transition temperature and contract the wire section and tension the tendon member to move the finger, and actuation of the gas source flows gas through the inlet port, into the tube, along the heated section of the wire in the tube and out the outlet port to quickly cool and lengthen the wire.

2. The controller as in claim 1 wherein said tube includes a loop section located between said tube ends; said wire includes a loop wire section in the loop section of the tube; said first electrical contact located on one side of the loop section of the wire and the second electrical contact located on the other side of the loop section of the wire.

3. The controller as in claim 1 wherein the said tube includes a coil section located between said tube ends; said wire includes a coiled wire section extending along the coiled section of the tube; and said first electrical contact is on one side of the coil section of the wire and the second electrical contact is located on the other side of the coiled section of the wire.

4. The controller as in claim 3 wherein said coiled sections of the tube and the wire are cylindrical.

5. The controller as in claim 3 wherein said coiled sections of said tube and wire are flat.

6. The controller as in claim 3 wherein said coiled tube is located in a housing defining an interior passage, an inflatable cuff located in said passage, wherein said housing may be mounted on the wrist of a wearer and held in place by inflating said cuff.

7. The controller as in claim 1 including a temperature sensor in the tube between the contacts, a valve between the gas source and the gas inlet port, and an operative connection between the sensor and the valve, wherein the sensor opens the valve to flow gas through the tube when the temperature of the wire is above the transition temperature.

8. The controller as in claim 1 wherein the electrical contacts are between or adjacent the inlet and outlet ports so that the gas flows along and cools the section of the wire.

9. A controller as in claim 1 wherein the second end of the tube is attached to a support member.

10. The method of moving a finger having a tendon member joined to one end of a shape memory alloy wire, the other end of the shape memory alloy wire mounted on a support member, the shape memory alloy wire extending along the interior of a dielectric tube, comprising the steps of:
a) flowing an electrical current through the shape memory alloy wire to heat the wire above a transition temperature, shrink the wire and tension the tendon member to actuate the finger; and
b) flowing a gas along the interior of the tube past the heated shape memory alloy wire to quickly cool the shape memory alloy wire below the transition temperature and lengthen the shape memory alloy wire.

11. The method of claim 10 including the step of:
c) after performing step b), again flowing electricity through the shape memory alloy wire to again shrink the wire, tension the tendon member and actuate the finger.
